(19) United States
(12) Patent Application Publication
(10) Pub. No.: US 2010/0055764 A1
(43) Pub. Date: Mar. 4, 2010

(54) CELL CULTURE APPARATUS AND METHOD

(76) Inventors: Gregory R. Martin, Acton, ME (US); Allison J. Tanner, Portsmouth, NH (US)

Correspondence Address: CORNING INCORPORATED, SP-TI-3-1, CORNING, NY, 14831, US

(21) Appl. No.: 12/199,357

(22) Filed: Aug. 27, 2008

(51) Int. Cl. C12N 1/00; C12M 3/06; C12M 3/00

(52) U.S. Cl. 435/243; 435/289.1; 435/297.1

(57) ABSTRACT

Cell culture apparatuses and methods for culturing cells include bags for culturing cells. The bags are folded and unfolded to control cell culture conditions. As the bags are folded, segmented sealed chambers for culturing cells are formed. As the bags are unfolded, the segments are unsealed allowing for exchange of fluid between the regions of the subchambers.

### FIELD

The present disclosure relates to apparatuses for culturing cells; more particularly to apparatuses that may contain a variable volume of fluid such as flexible, bag-type vessels.

### BACKGROUND

Currently, there is no commercially available device that readily permits expansion from smaller to larger scale cell culture, that is easy to operate and manufacture, and significantly reduces the disposable waste volume.

Current systems containing expandable bags allow for expansion from smaller to larger scale culture. However, as the scale increases, oxygenating cells becomes more difficult, often requiring rocking or sparging. At large volumes, rocking may cause cell shearing and foaming problems.

Another perceived problem with cells grown in bags is the tendency to kink and cut off flow in certain segments of the bag, isolating cells from media and gas exchange.

### BRIEF SUMMARY

Many of the devices and methods described herein take advantage of the kinking and cutting off of flow that can occur in bag-type cell culture apparatuses. A cell culture bag may be sufficiently folded to seal a major chamber of the bag into separate subchambers for culturing or may be sufficiently unfolded to allow cell culture media or other fluid to flow between the regions of the separate subchambers.

In various embodiments, a method for culturing cells includes (i) introducing culture media and cells into a flexible major culture chamber of a cell culture bag, (ii) folding the bag to form first and second subchambers from the major chamber, and (iii) incubating the cells in the first and second subchambers.

In various embodiments, a cell culture apparatus includes a housing and a bag for culturing cells. The bag forms a major cell culture chamber and is disposed in the housing such that a second portion of the bag is folded relative to a first portion of the bag, thereby dividing the major chamber into first and second subchambers. The first portion of the bag forms the first subchamber and the second portion of the bag forms the second subchamber. The bag further includes a curved portion between the first and the second portions. The curved portion of the bag includes a first wall and an opposing second wall. A portion the first wall is moveable relative to a portion of the second wall from a position in contact with the second wall to fluidly seal the first subchamber from the second subchamber to a position away from the second wall to allow cell culture fluid to flow between the first and second subchambers.

In various embodiments, a cell culture apparatus includes a housing, a bag for culturing cells, and a pivotable element. The bag forms a major cell culture chamber and is disposed in the housing such that a second portion of the bag is foldable and unfoldable relative to a first portion of the bag. When the bag is sufficiently folded (i) the major chamber is divided into a first subchamber formed by the first portion of the bag and a second subchamber formed from the second portion of the bag, and (ii) the bag comprises a curved region disposed between the first and second portions of the bag. The pivotable element is aligned with the curved region of the bag. Pivoting of the pivotable element causes the bag to fold or unfold.

In various embodiments, tracheal elements are employed to promote exchange of gas between cell culture media within the bag and the environment exterior to the bag. Use of such tracheal members can, in many circumstances, eliminate the need for rocking, sparging, or stirring often required for prior culture apparatuses.

With apparatuses as described herein, as cells in the culture proliferate, the culture may be readily and simply expanded by relaxing segmentation by sufficiently unfolding to allow introduction of additional cell culture medium into the bag. Another potential advantage of the apparatuses described herein relates to the reduction in disposal of material of construction at the end of the culture period, as in some embodiments, the housing and other components may be re-usable. Further, manufacturing complexity may be greatly reduced relative to stirred vessels. These and other advantages of the various embodiments described herein will readily understood from the following detailed description when read in conjunction with the accompanying drawings.

### BRIEF DESCRIPTION OF THE DRAWINGS

FIGS. 1A-E are schematic drawings of longitudinal cross-sections of a cell culture bag or portions thereof.

FIGS. 2A-C are schematic drawings of longitudinal cross-sections of a cell culture bag.

FIGS. 3-4 are schematic drawings of longitudinal cross-sections of cell culture bags and tracheal members.

FIGS. 5A-B are schematic drawings of side views of cell culture apparatuses. When discussed with regard to FIG. 6B, the drawings of FIGS. 5A and 5B are top-down views.

FIGS. 6A-B are schematic drawings of perspective views of a cell culture apparatus.

FIG. 7 is a schematic drawing of a longitudinal cross-section of a pulling member.

FIGS. 8A-B are schematic drawings of a top-down view of unfolded panels and a cell culture bag.

FIGS. 8C-D are schematic drawings of a side view of panels partially folded (B) or completely folded (C).

FIGS. 8E-F are schematic drawings of a top-down view of unfolded panels and a cell culture bag.

FIGS. 9A-B are schematic drawings of an exploded top view (A) and top view (B) of a cell culture bag disposed on an array of panels.

FIGS. 10A-B are schematic drawings of an exploded side view (A) and side view (B) of a cell culture bag interleaved between spindles.

FIGS. 11A-C are schematic drawings of side views of cross-sections of an apparatus into which a cell culture bag disposed about spindles may be placed.

FIGS. 12A-B are schematic drawings of overhead views of the apparatus of FIGS. 11A-C in which a cell culture bag disposed about spindles is be placed.

FIG. 13 is a schematic drawing of a front view of components of an apparatus depicted in FIGS. 12A-B.

FIG. 14 is a schematic drawing of a longitudinal cross-section of a cell culture bag including a membrane.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The present disclosure describes, inter alia, cell culture apparatuses that allow for folding and unfolding of cell culture bags. As the bags are folded, segmented sealed chambers for culturing cells are formed. As the bags are unfolded, the segments are unsealed allowing for exchange of fluid between the regions of the subchambers.

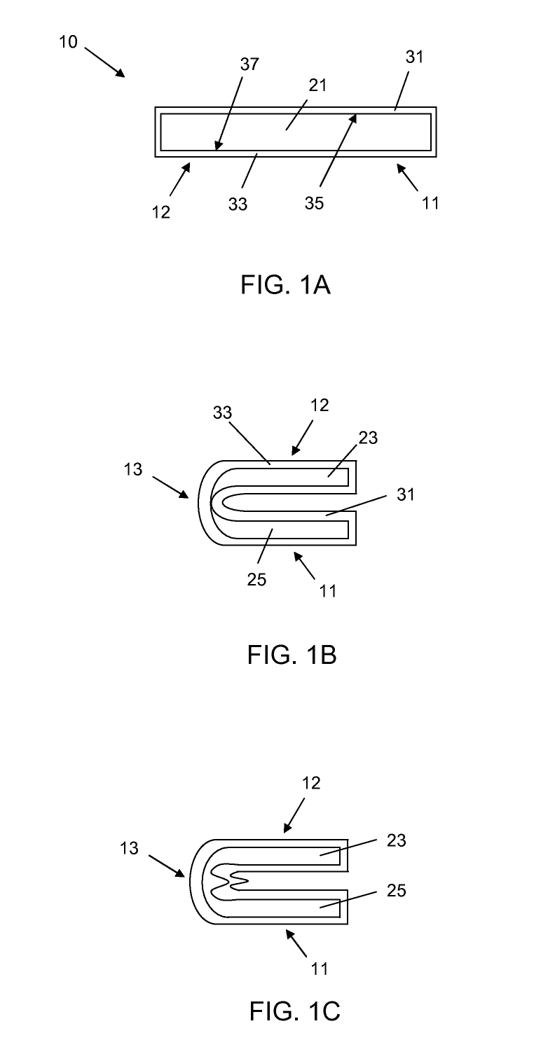

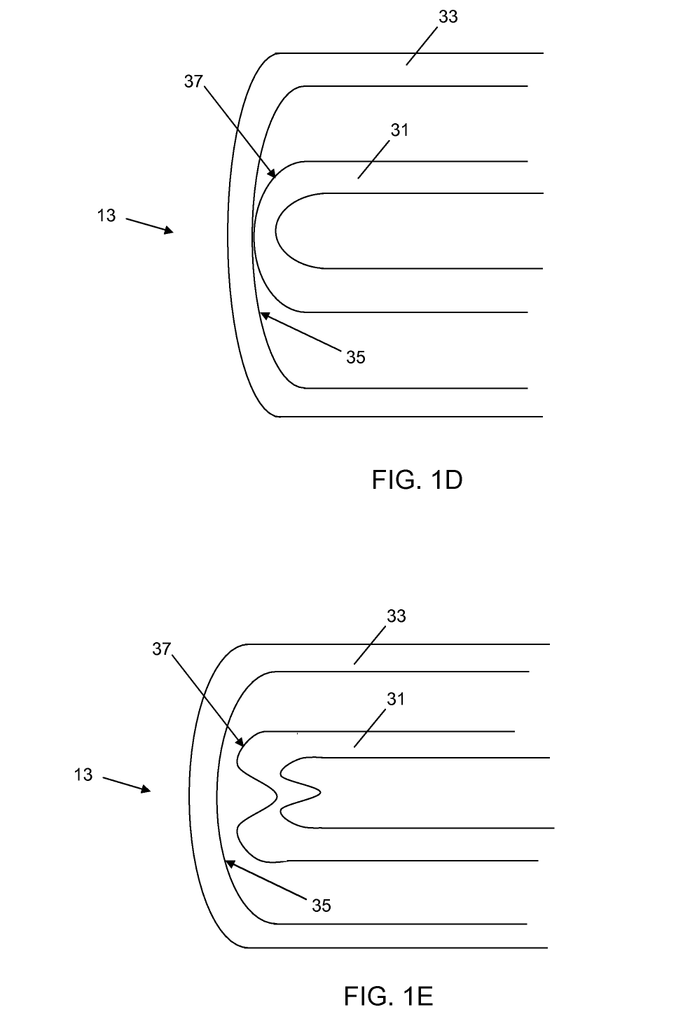

Referring to FIGS. 1A-E schematic longitudinal cross-sectional views of a cell culture bag are shown. As shown in FIG. 1A, the bag 10 forms a major chamber 21 for culturing cells. The volume of the chamber 21 is variable and can change depending on the volume of cell culture media in the chamber 21. The bag 10 includes a first wall 31 and an opposing second wall 33. The first wall 31 has an inner surface 35 and an outer surface, and the second wall 33 has an inner surface 37 and an outer surface. In FIG. 1B, a second portion 12 of the bag 10 of FIG. 1A is folded back on a first portion 11 of the bag creating a curved portion 13 between the first 11 and second 12 portions of the bag. The second portion 12 is folded back on the first portion 11 of the bag such that the major chamber is divided into first 23 and second 25 subchambers. The first 23 and second 25 subchambers may be fluidly sealed from each other by an occlusion in the curved region 13. While the curved region 13 is shown as a smooth curve, it will be understood that the curve may be abrupt or angular. The curved region 13 may be shaped in any configuration that provides a reversible fluid seal between the first and second subchambers 23, 25. As shown in FIG. 1C, a portion of the first wall in the curved region 13 may be pulled or moved relative to the second wall to allow cell culture media to flow between the region of the first subchamber 23 and the region of the second subchamber 25. FIGS. 1D and 1E are close-up views of the curved regions shown in FIGS. 1B and 1C, respectively. As shown in FIGS. 1D-E, the first wall 31 of the bag is moveable from a sealed position in which a portion of the inner surface 35 of the first wall 31 is in close proximity to or in contact with a portion of the inner surface 37 of the second wall 33 (see FIG. ID) to an open position in which sufficient space is provided between the inner surface 35 of the first wall 31 and the inner surface 37 of the second wall 33 to allow cell culture media to flow between the region of the first subchamber 23 and the region of the second subchamber 25.

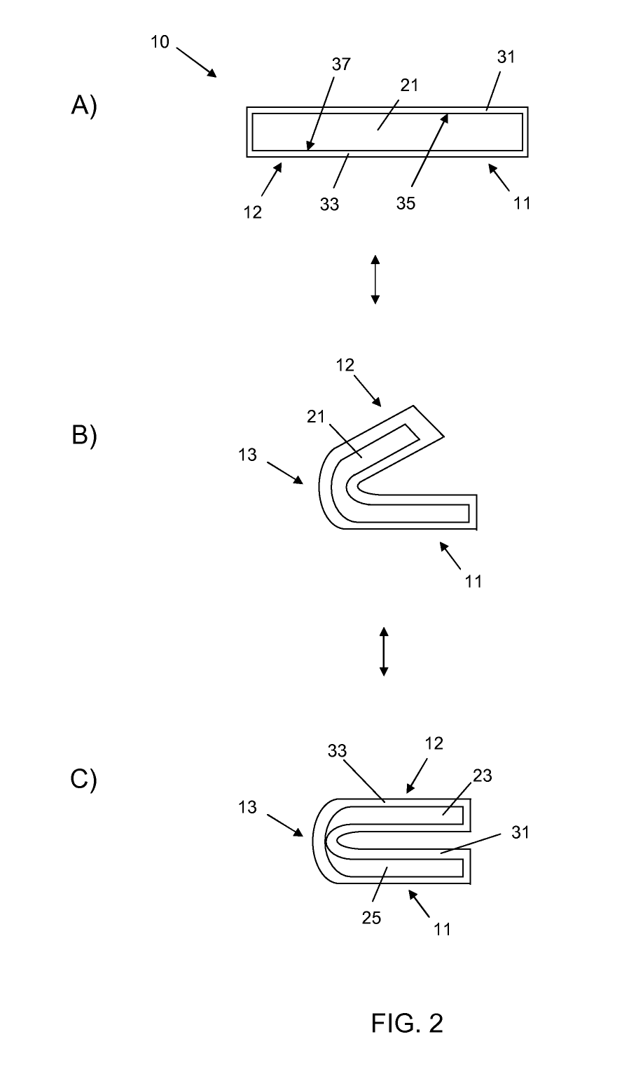

Referring now to FIGS. 2A-C, an alternative mechanism for sealing and unsealing the first subchamber 23 relative to the second subchamber 25 is shown. The bag 10 shown in FIG. 2A is the same as the bag 10 shown in FIG. 1A and includes a major chamber 21, a first wall 31 and an opposing second wall 33. The first wall 31 has an inner surface 35 and an outer surface, and the second wall 33 has an inner surface 37 and an outer surface. As shown in FIG. 2B, a second portion 12 of the bag 10 is folded back on a first portion 11 of the bag creating a curved portion 13 between the first 11 and second 12 portions of the bag. In FIG. 2B, the bag is not folded back on itself to a sufficient degree to cause dividing of the major chamber 21 into first an second subchambers. As shown in FIG. 2C, further folding of the first portion of the bag 10 back on itself causes the major chamber to form first 23 and second 25 subchambers that are fluidly sealed from one another due to a portion of the inner surface of the first wall contacting a portion of the inner surface of the second wall at the curved portion 13. As depicted, the bag may be folded or unfolded to seal or allow fluid to flow between the region of the first subchamber 23 and the region of the second subchamber 25.

A bag 10, as described herein, may be formed into any suitable form for culturing cells, such as an inflatable pouch, bladder, bag, tube, or the like. Bags 10 may be formed from films by heat sealing, laser welding, application of adhesive, or any other method known in the art of inflatable bag making. Walls or portions thereof of a bag 10 may have a thickness that allows for efficient transfer of gas across the wall. It will be understood that desired thickness may vary depending on the material from which the wall is formed. By way of example, the wall or film forming the wall may be between about 0.02 millimeters and 0.8 millimeters thick. Prior to sealing or forming the bag, it may be desirable to treat or coat that portion of the material in which cells will be cultured once formed. Alternatively, the bag 10 may be treated after it is formed. The treatment or coating may facilitate cell culture. Treatment may be accomplished by any number of methods known in the art which include plasma discharge, corona discharge, gas plasma discharge, ion bombardment, ionizing radiation, and high intensity UV light. Coatings can be introduced by any suitable method known in the art including printing, spraying, condensation, radiant energy, ionization techniques or dipping. The coatings may then provide either covalent or non-covalent attachment sites. Such sites can be used to attach moities, such as cell culture components (e.g., proteins that facilitate growth or adhesion). Further, the coatings may also be used to enhance the attachment of cells (e.g., polylysine).

Bag 10 may be made of any material suitable for culturing cells. In various embodiments, the bag 10 is formed of optically transparent material to allow visual inspection of cells cultured in the bag 10. Preferably, the bag 10 is gas permeable to allow exchange of gasses across the bag as cells are being cultured. Examples of optically transparent, gas permeable materials that may be used to form the bag 10 include polystyrene, polycarbonate, ethylene vinyl acetate, polysulfone, polymethylpentene, polytetrafluoroethylene (PTFE) or compatible fluoropolymer, a silicone rubber or copolymer, poly(styrene-butadiene-styrene), or polyolefin, such as polyethylene or polypropylene, or combinations of these materials.

A bag 10, as described herein, may be configured to hold any suitatable volume of cell culture medium and cells. For example, a given bag may be configured to hold about 100 milliliters or less, while other bags may be configured to hold more than about 1000 liters. In various embodiments, the bag is expandable to a volume of between about 0.1 m/cm² and about 0.5 ml/cm2. For example, the bag may be expandable to a volume of between about 0.2 ml/cm² and about 0.3 ml/cm².

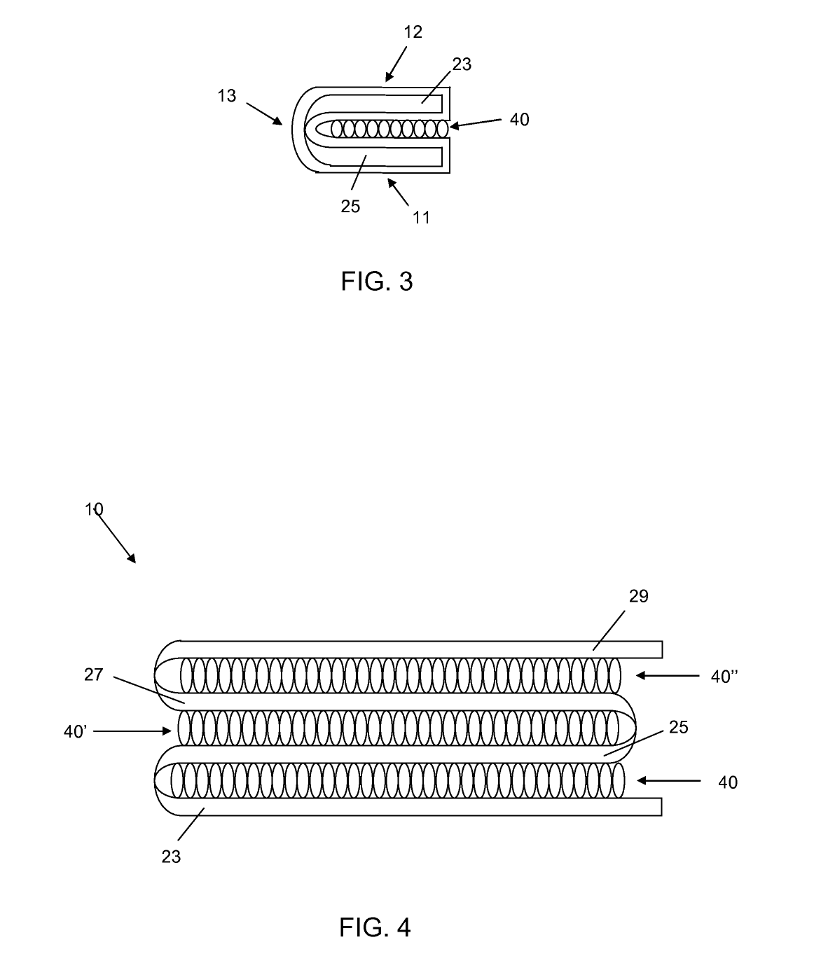

Referring now to FIG. 3, a second part 12 of a bag is shown folded back on a first portion 11 of the bag, forming first 23 and second 25 subchambers. A tracheal element 40 is disposed between the first portion 11 of the bag and the second portion 12 that is folded back on the first portion 11. Tracheal member 40 is configured to create air space for gas exchange across the bag 10. As such gas in the cell culture media within a chamber or subchamber 23, 25 may be exchanged with gas external to the bag 10 to maintain desirable culture conditions in the chamber or subchamber 23, 25. Preferably, tracheal members 40 are located on both sides of the bag 10 (e.g., the bag 10 is disposed between two tracheal elements 40) to increase the surface area of air space in contact with the bag 10. However, it will be understood that a tracheal member 40 may be disposed only on one side of the bag 10 or no tracheal member 40 may be employed if sufficient exchange of air across the bag 10 is achieved for the intended cell culture purpose. Tracheal elements 40 may take any suitable form, such as a woven mesh or plates with ridges, bumps, troughs or the like. Such tracheal members 40 may be molded or otherwise formed of nearly any suitable material, such as polymeric material. Examples of suitable polymeric materials include polystyrene, polycarbonate, ethylene vinyl acetate, polysulfone, polymethylpentene, polytetrafluoroethylene (PTFE) or compatible fluoropolymer, silicone rubber or copolymer, poly(styrene-butadiene-styrene), or polyolefin, such as polyethylene or polypropylene, or the like, or combinations of thereof. In some embodiments, the tracheal member 40 is a mesh stocking formed from a suitable polymeric material, such as polyester, polyamide or the like.

As shown in FIG. 4, a bag 10 may be folded back upon itself more than once to create more than two subchambers 23, 25, 27, 29 for culturing cells. A tracheal element 40, 40′, 40″ may be disposed between each portion of the bag that is folded back on itself to allow air flow and gas exchange with the subchambers 23, 25, 27, 29. Tracheal element 40 may be formed from or affixed to or adhered to bag 10.

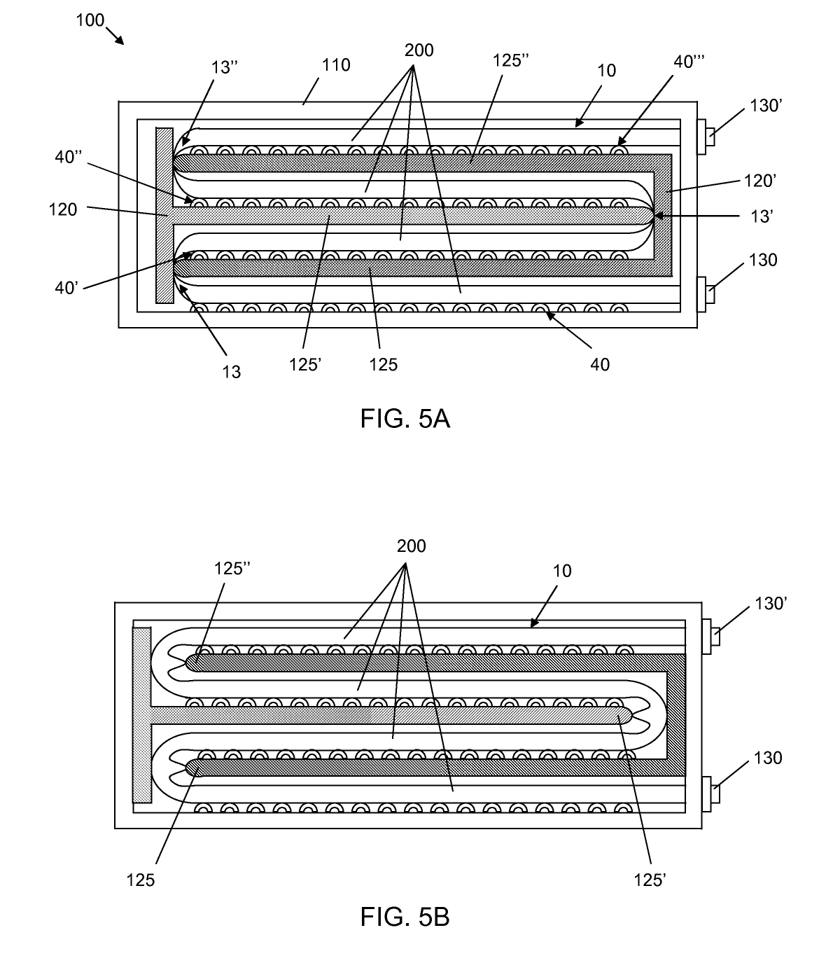

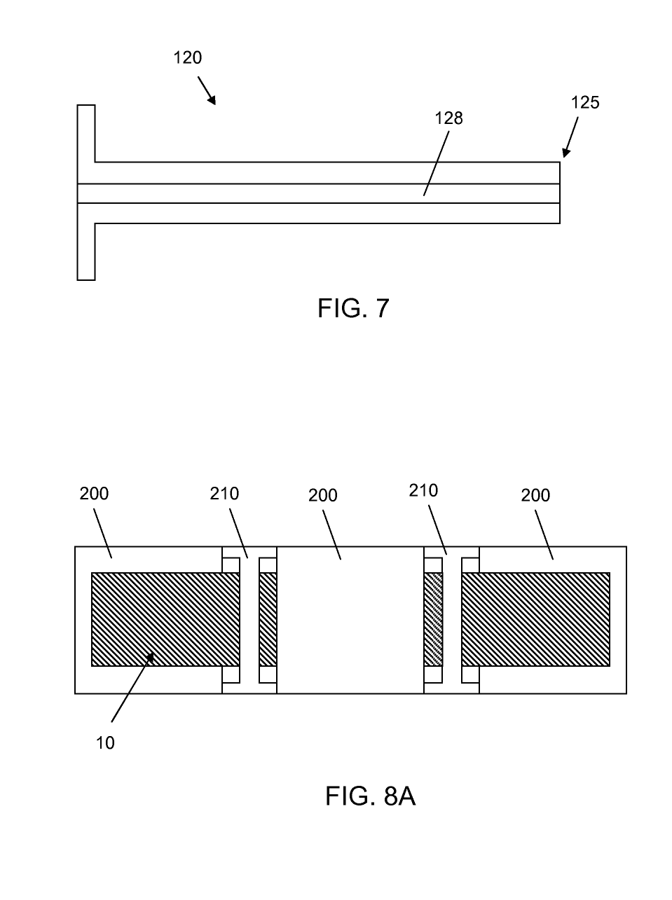

Referring now to FIGS. 5A-B, an apparatus 100 for culturing cells is depicted. The apparatus 100 includes a housing 110 and a bag 10 disposed in the housing and folded back on itself multiple times to form multiple subchambers 200, each fluidly sealed from the other (FIG. 5A). The apparatus 100 further includes pulling members 120, 120′ configured and positioned to pull a portion of a wall of the bag 10 in a curved portion 13, 13′, 13″ relative to an opposing wall to allow fluid to flow between adjacent subchambers 200 separated by the curved portion 13, 13′, 13″ (FIG. 5B). A pulling member may have one (120) or more (120′) distal portions 125, 125′, 125″ located in proximity to a wall of the bag 10 in the curved portion 13, 13′, 13″. For example, a distal portion 125, 125′, 125″ may be in contact with a wall of a curved portion 13, 13′, 13″ or may be located close enough that a vacuum pulled through a lumen extending through the distal portion 125, 125′, 125″ of the pulling member 120, 120′ could cause the wall in the curved region 13, 13′, 13″ to move relative to the opposing wall to allow fluid to flow. In various embodiments, distal portion 125, 125′, 125″ is bonded or affixed to a wall of the curved region 13, 13′, 13″. For example, distal portion 125, 125′, 125″ may be adhered or grippingly engage the wall. In other embodiments, such as depicted in FIG. 7 and described in more detail below, a pulling member 120, 120′ may include a lumen extending through the distal portion 125, 125′, 125″ to allow a vacuum to be pulled through the lumen to move the wall in the curved region 13, 13′, 13″ relative to the opposing wall. As shown in FIGS. 5A-B, pulling members 120, 120′ may be moveable in housing to allow the wall of the bag 10 to be pulled. Alternatively, distal portion 120, 120′ may be located away from the wall to be pulled and a vacuum or other element inserted into a lumen of pulling member 120, 120′ maybe used to pull the wall of the bag 10.

In various embodiments, the pulling members 120, 120′ may be pushed against the wall in the curved region 13, 13′, 13″ to facilitate sealing of adjacent subchambers 200. However, with many bags 10 a sufficient seal can be formed by folding the bag 10 back upon itself

In the embodiment depicted in FIGS. 5A-B, apparatus includes tracheal elements 40, 40′, 40″, 40′″ disposed on arms of pulling members 120, 120′. In various embodiments, tracheal elements 40, 40′, 40″, 40′″ are formed from pulling members 120, 120′. In numerous embodiments, tracheal members 40, 40′, 40″, 40′″ are bonded or affixed to pulling members 120, 120′. Of course tracheal members may be separate elements or bonded or affixed to bag 10. While shown on only one side of pulling member 120, 120′, tracheal elements 40, 40′, 40″, 40′″ may be disposed on both the top and bottom surface of the arms of pulling members 120, 120′.

In various embodiments, the bag 10 is disposable and the housing 110 or pulling members 120, 120′ are re-usable. A replacement bag 10 may be placed in housing and weaved around pulling members 120, 120′ as shown in FIGS. 5A-B. Pulling members 120, 120′, if moveable, may be moved to a position indicated in FIG. 5B to facilitate insertion of the replacement bag into the housing 110. Following insertion of replacement bag, the pulling members 120, 120′, if moveable, may be moved to a position as illustrated in FIG. 5A and distal portion 125, 125′, 125″ of a pulling member 120, 120′ may be bonded or affixed to a wall of the bag 10 in a curved region 13, 13′, 13″ as appropriate. Pulling members 120, 120′ may be moved by any suitable mechanism. For example, pulling members 120, 120′ may be moved manually, via a pneumatic mechanical slide system, or the like.

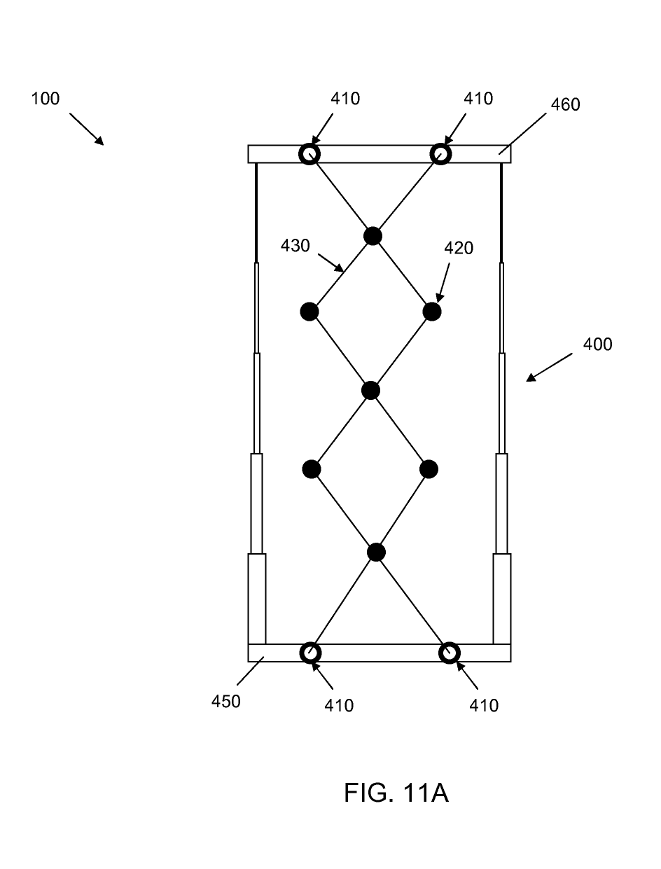

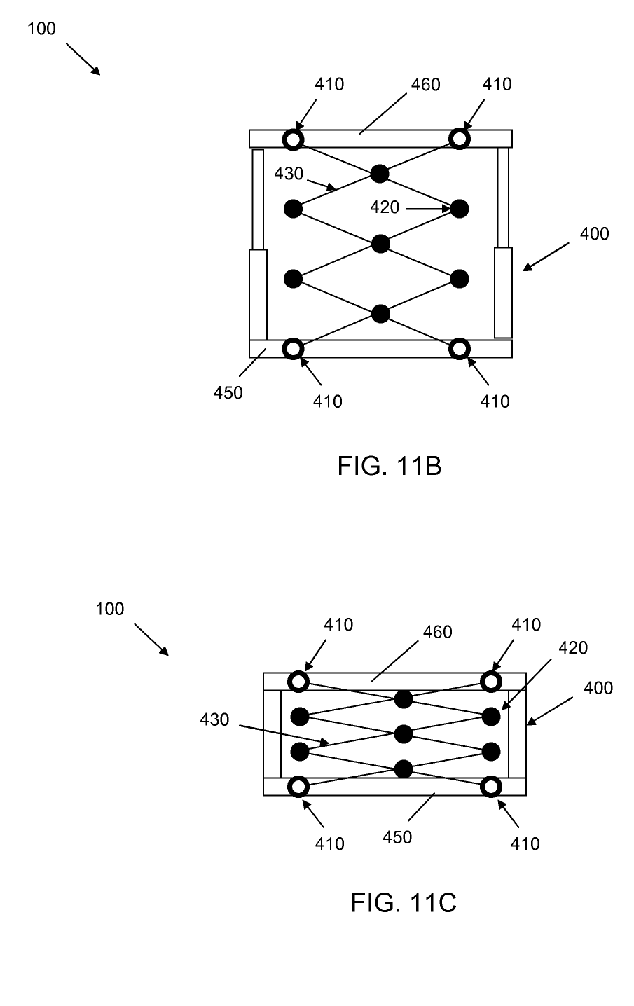

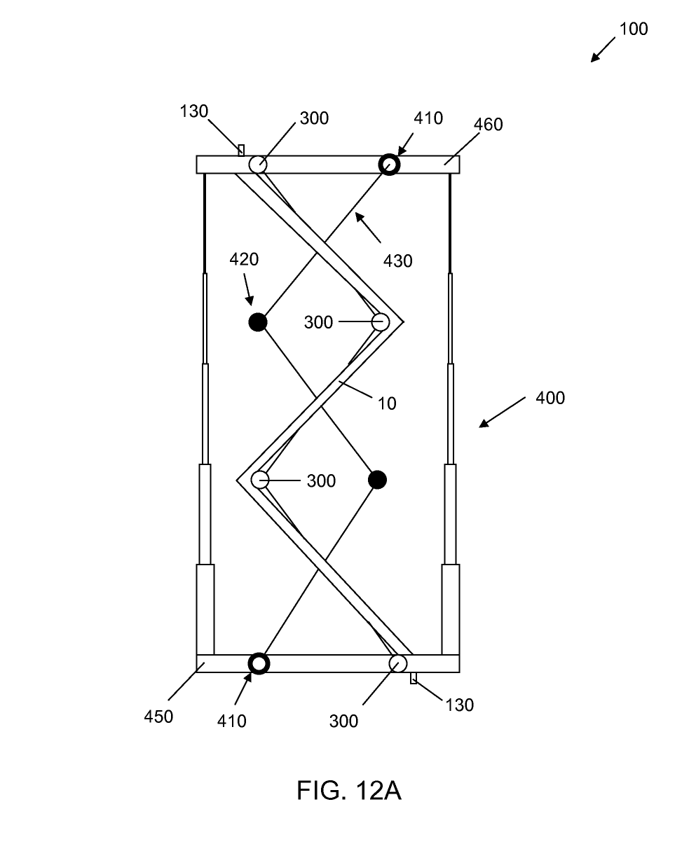

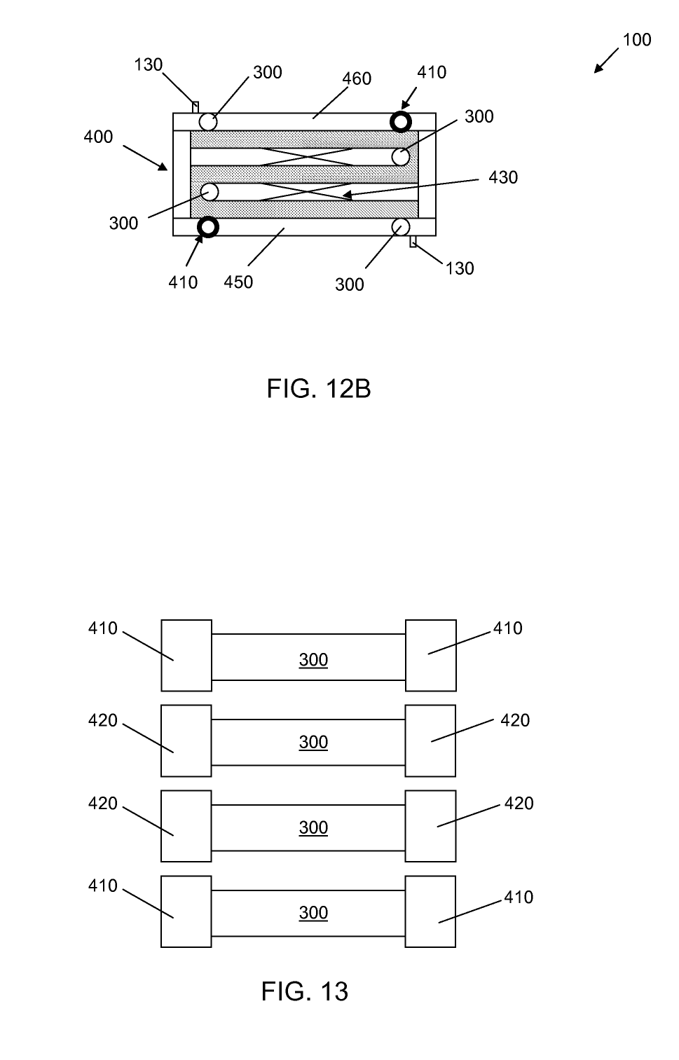

Housing 110 and pulling member 120, 120″ may be made of any suitable material, such as stainless steel, polystyrene, polyethylene, polycarbonate, ethylene vinyl acetate, polypropylene, polysulfone, polymethylpentene, polytetrafluoroethylene (PTFE) or compatible fluoropolymer, a silicone rubber or copolymer, poly(styrene-butadiene-styrene), or the like, or a combination thereof. If housing 110 or pulling member 120, 120″ are intended to be reusable, housing 110 or pulling member 120, 120″ are preferably made of material that can withstand repeated rounds of washing or autoclaving. In general, housing 110 should be sufficiently thick to maintain sufficient rigidity. For example, housing 110 may have a thickness of between about 1 millimeter and about 2.5 millimeter or more. Housing 110 may have any suitable dimension, which may depend on the volume capacity of bag 10. In some embodiments, the dimensions of housing 110 are variable (see, e.g., FIGS. 11 and 12).

As shown in FIGS. 5A-B, bag 10 includes one or more ports 130, 130′ for introducing or removing cell culture or other media from the bag 10. A port 130, 130′ may be fluidly sealed with a bag 10 through any suitable mechanism, such as, for example, heat sealing or RF welding. In some embodiments, such as depicted in FIGS. 5A-B, bag 10 includes two ports 130, 130′. The inclusion of more than one port can facilitate filling or emptying of bag 10 by allowing venting or pressure equilibration. Caps, septums, valves, or the like (not shown) may be used to seal the ports 130, 130′ when media is not being added or removed from the bag 10. In some embodiments, ports 130, 130′ form a part of housing 110 and the bag includes ports configured to sealingly couple to the housing ports; e.g., through connection fittings, such as complementary threads for screwing, quick lock connectors, or the like.

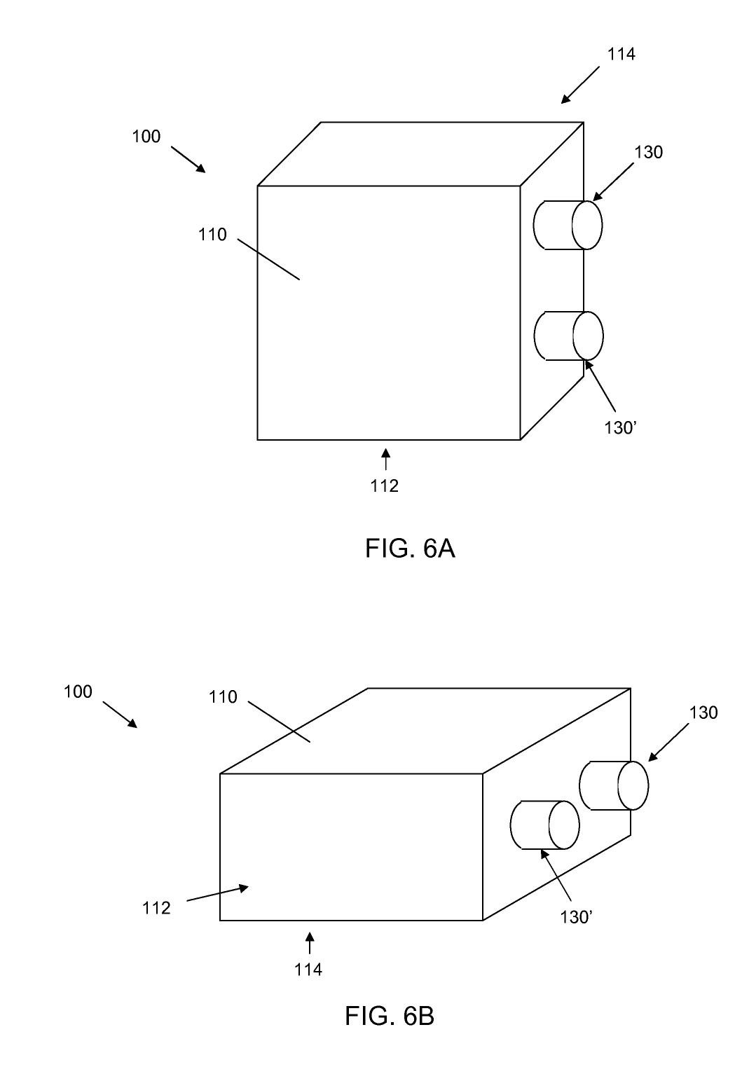

Still with reference to FIGS. 5A-B and referring to FIGS. 6A-B, culture fluid or cells may be introduced into the bag 10 through a port 130, 130′ while fluid is capable of flowing between the regions of adjacent subchambers 200 (FIG. 5B). In various embodiments, culture fluid or cells are introduced into bag 10 while apparatus 100 is positioned on a first surface 114 of housing 110 (the orientation of FIG. 6B corresponds to the orientation of FIGS. 5A-B, if FIG. 5A-B are considered a top-down view). Following introduction of fluid into the bag 10, the fluid (e.g., cell culture medium and cells) may be allowed to distribute evenly throughout the bag 10 prior to releasing the pulling force to allow sealing of subchambers 200. In this manner, each subchamber 200 may contain approximately equal amounts of cell culture fluid and cells. Further, introducing fluid into the bag 10 while the apparatus 10 is on its “side” (FIG. 6B) as opposed to “upright” (FIG. 6B) reduces problems with unequal loading of subchambers 200 due to hydrostatic forces. For culturing cells, the apparatus 10 may then be rotated ninety degrees such that apparatus rests on a second surface 112 that is orthogonal and adjacent to the first surface 114 (FIG. 6A: the orientation of FIG. 6A corresponds to the orientation of FIGS. 5A-B, if FIG. 5A-B are considered a side view).

While side surface in FIG. 6A (top surface in FIG. 6B) of housing 110 is shown as enclosed, the surface may be open, fully or partially, to allow for free exchange of gasses external to housing and within culture bag along tracheal elements. Alternatively, openings (not shown) may be created in the side surface to which a gas inlet and outlet line may be coupled to control the gas environment within the housing 110.

Referring now to FIG. 7, a longitudinal cross-section of a pulling member 120 is shown. The depicted pulling member 120 has a proximal end and a distal end 125 and a lumen 128 extending through the pulling member 120 from the proximal end to the distal end 125. A vacuum pump may be operably coupled to proximal end of lumen 128 so that a wall of the bag in the curved region between the subchambers may be sucked towards the distal end 125 or attach to the distal end 125 of the pulling member 125 to allow fluid to flow between subchambers (e.g. as discussed above with regard to FIG. 1 and FIG. 5). Alternatively, a separate element (not shown) slidably disposable in lumen 128 may be extendable from lumen 128 and may be used to engage and pull a wall of the bag. Such a configuration may be desirable when the pulling elements are not moveable (e.g., permanently positioned in a configuration as shown in FIG. 5B).

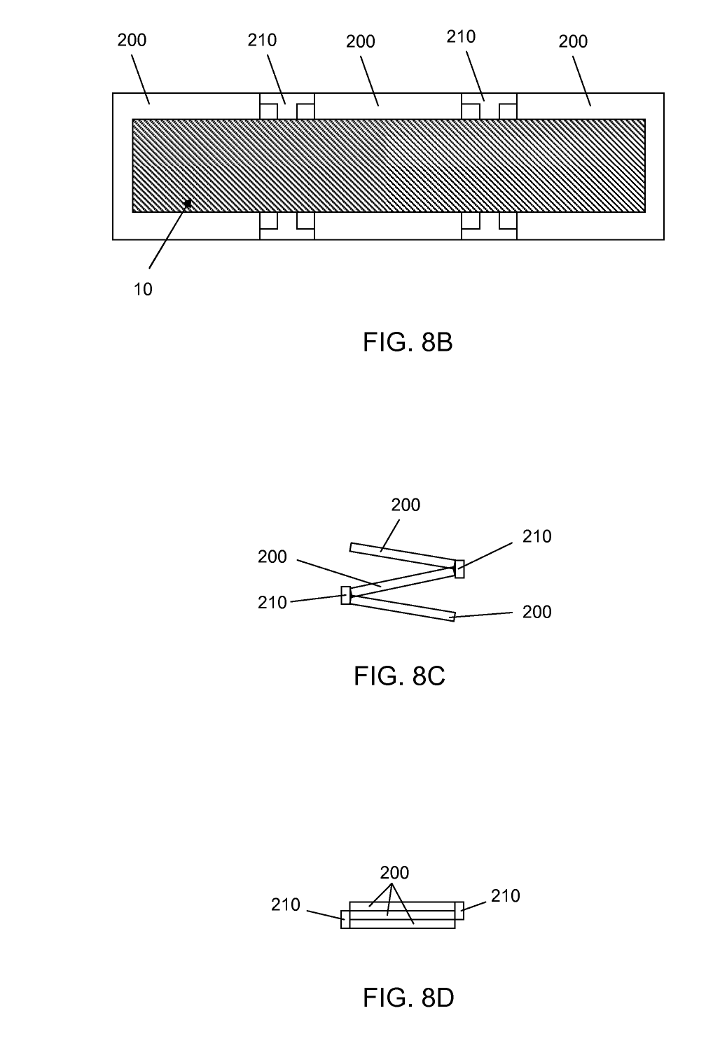

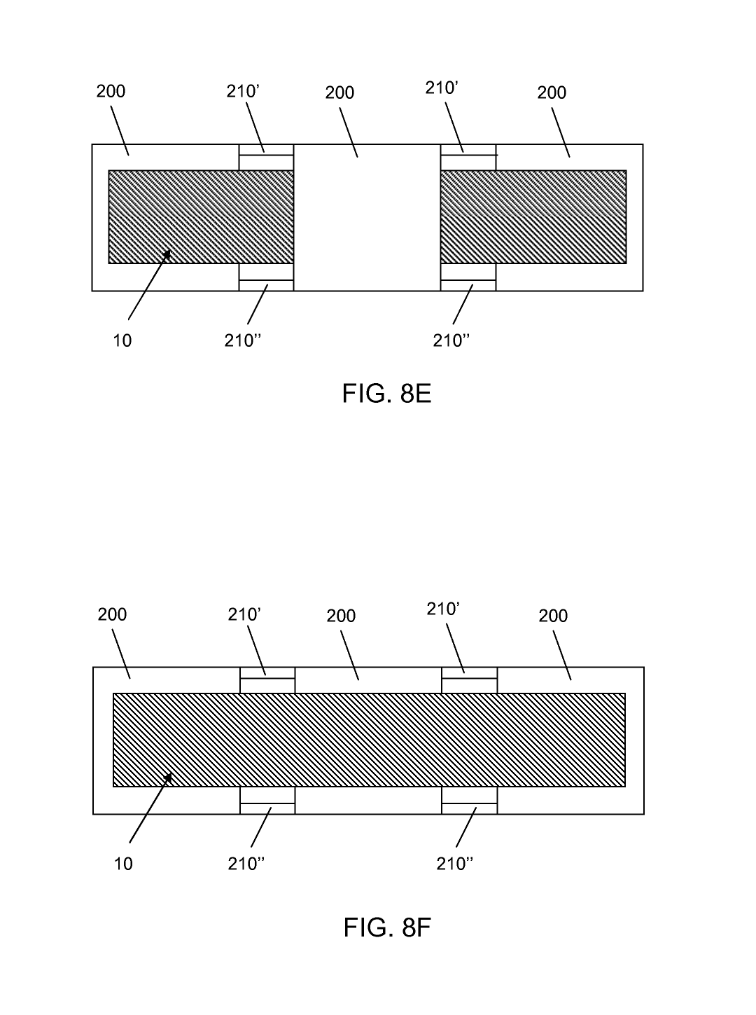

Referring now to FIGS. 8A-D, bag 10 is weaved between foldable panels 200 (8A) or overlaid on foldable panels 200 (8B). Adjacent panels 200 in the depicted embodiment are coupled via pivotavle elements 210, such as hinges. The panels 200 may be folded and unfolded to allow sealing and unsealing of subchambers of bag 10; e.g. as depicted in FIG. 2. The bag 10 may be loosely disposed on panels 200 or bonded or affixed to one or more panels 200.

In the embodiments depicted in FIGS. 8C-D, the panels 200 are folded in alternating inward/outward bend orientation. The pivotable elements 210 may be configured to provide for such a folding orientation. Of course, the panels 200 may be folded in any suitable manner. In various embodiments (not shown), panels 200 are separate and may be folded in a suitable manner without the use of hinges. The panels 200 and bag 10 may be disposed within a housing (not shown).

Referring now to FIGS. 8E-F, which are substantially the same as FIGS. 8A-B, except that the center portion or spindle of pivotable element in FIGS. 8A-B is missing in FIGS. 8E-F. In FIGS. 8E-F, hinges 210′ and 210″ together form a pivotable element that is aligned with a portion of the bag 10 that will be curved when the bag 10 is folded.

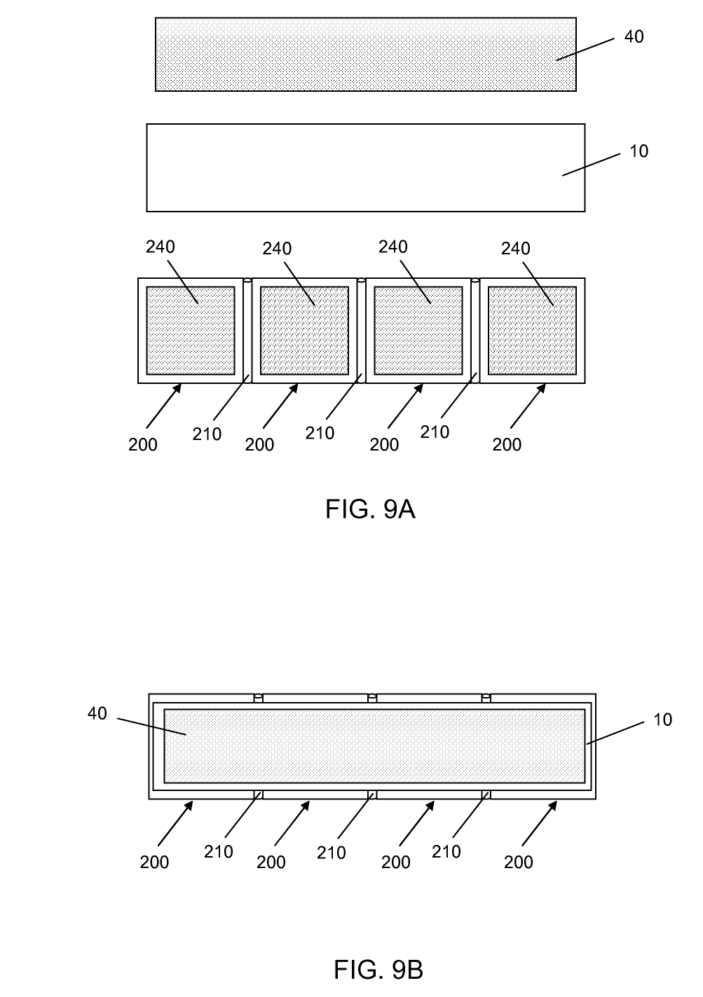

Referring now to FIGS. 9A-B, an exploded top view (A) and top view (B) of a bag 10 disposed on an array of panels is shown. As in FIGS. 8A-B, adjacent panels 200 are connected by hinges 210 and may be folded or unfolded in a suitable manner to seal or unseal subchambers of bag 10. The panels 200 include tracheal regions 240 that allow for air to flow along the surface of a bag 10 disposed on a panel 200, allowing for exchange of gasses between a cell culture chamber of bag 10 and the environment outside the bag 10. The tracheal region 240 may be a tracheal element bonded or adhered to panel 240. Alternatively, tracheal region 240 may be formed from panel 200, e.g. by molding or thermoforming. While tracheal region 240 is shown as occupying a portion of the top surface of panel 200, it will be understood that tracheal region 240 may occupy all or substantially all of the top surface of panel 200. The bottom surface (not shown) of panel 200 may also include a tracheal region. In the embodiment depicted in FIG. 9B, bag 10 is disposed on panels 200 and in contact with tracheal regions and a separate flexible tracheal member 40 is disposed on bag 10.

Panels 200 and pivotable elements 210 may be made of any suitable material. For example, panels 200 and pivotable elements 210 may be made of stainless steel, polystyrene, polyethylene, polycarbonate, ethylene vinyl acetate, polypropylene, polysulfone, polymethylpentene, polytetrafluoroethylene (PTFE) or compatible fluoropolymer, a silicone rubber or copolymer, poly(styrene-butadiene-styrene), or the like, or a combination thereof.

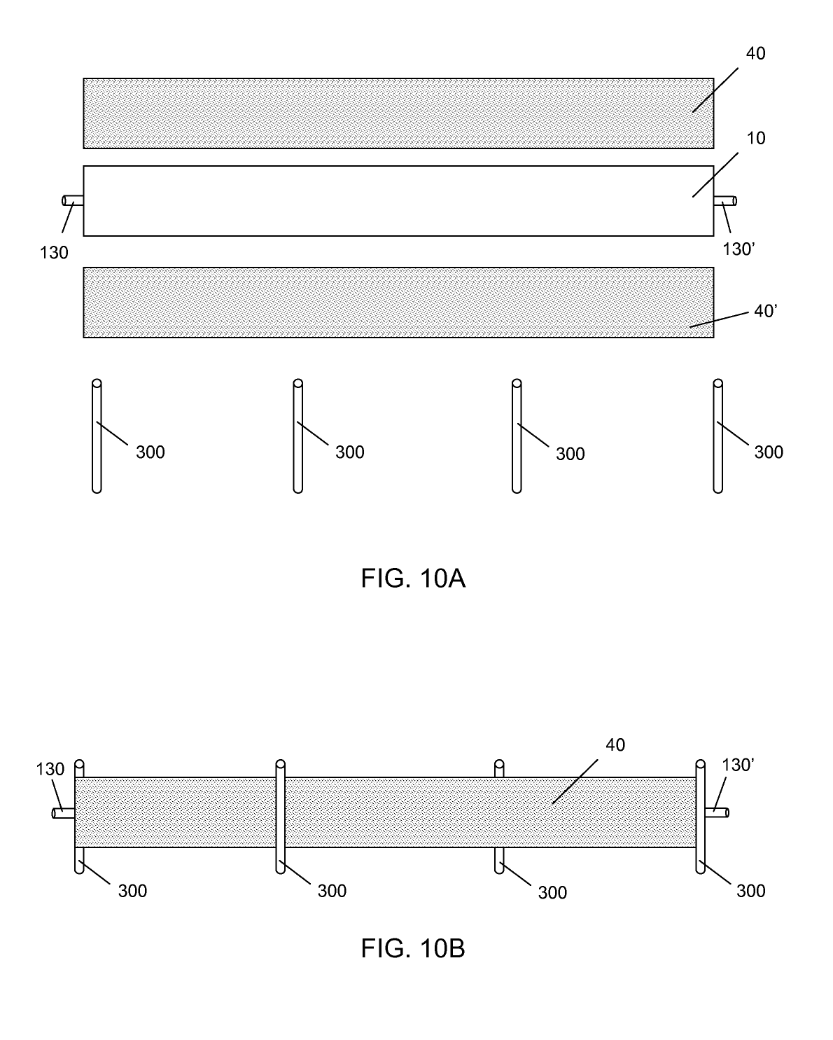

Referring now to FIGS. 10A-B, an exploded side view (A) and side view (B) of a bag 10 interleaved between spindles 300 is shown. The bag 10 is disposed between flexible tracheal members 40, 40′, such as a polymer fiber mesh. In some embodiments, the bag 10 is disposed in a mesh stocking, which serves as a tracheal member surrounding the bag. The bag 10 includes ports 130, 130′ to provide access to the cell culture chamber within the bag 10. The bag 10 or tracheal member 40, 40′ may be bonded or affixed to one or more spindles 300. The bag 10 may be folded or unfolded around a spindle 300 to seal or unseal adjacent subchambers; e.g. as described with regard to FIG. 2. The spindles may be of any suitable material, such as stainless steel, polystyrene, polyethylene, polycarbonate, ethylene vinyl acetate, polypropylene, polysulfone, polymethylpentene, polytetrafluoroethylene (PTFE) or compatible fluoropolymer, a silicone rubber or copolymer, poly(styrene-butadiene-styrene), or the like, or a combination thereof

Referring now to FIGS. 11A-C, side views of cross-sections of an apparatus 100 into which a bag disposed about spindles may be placed is shown. The apparatus 100 includes telescoping side walls 400 that are expandable and retractable, and bottom 450 and top 460 walls. The bottom 450 and top 460 walls include rails (not shown) for retaining and guiding wheels 410. The wheels 410 may be configured to accommodate insertion of a spindle. An accordion-type expandable and collapsible structure is disposed between the telescoping side walls 400. The structure includes an array of pivot elements, such as hinges 420, at least some of which are configured to accommodate insertions of a spindle, and rails 430 pivotably attached to hinges 420. Some of the rails 430 are pivotably attached to the wheels 410. As the side walls 400 are collapsed (compare FIG. 11A to 11B or FIG. 11B to 11C), the wheels move outwardly in rail (not shown) of top 460 and bottom 450 walls and the accordion-type structure collapses. Components of the apparatus 100 may be made of any suitable material. For example, the wheels 410, rails 430, pivotable elements 420, telescoping side walls 400, bottom wall 450, and top wall 460 may be made of stainless steel, polystyrene, polyethylene, polycarbonate, ethylene vinyl acetate, polypropylene, polysulfone, polymethylpentene, polytetrafluoroethylene (PTFE) or compatible fluoropolymer, a silicone rubber or copolymer, poly(styrene-butadiene-styrene), or the like, or a combination thereof. The side walls 400, and thus accordion-type structure, may be expanded and retracted manually or via machined mechanism, such as pneumatically, and may be positioned to expand horizontally or vertically.

Referring now to FIGS. 12A-B, overhead-views of the apparatus 100 depicted in FIGS. 11A and 11C are shown with a bag 10 disposed about spindles 300 are shown. The spindles 300 are inserted into or otherwise operably coupled or connected to pivotable elements 420. As telescoping sidewalls 400 are retracted or contracted (compare FIG. 12A to 12B), bag 10 is folded and adjacent subchambers are fluidly sealed from each other. As telescoping sidewalls 400 are extended, bag 10 is unfolded allowing fluid to flow between regions of adjacent subchambers of bag 10, e.g. as described with regard to FIG. 2.

While FIGS. 11-12 show only one accordion-type expandable and collapsible structure, apparatus 100 may include such a structure on both sides of spindle 300, where spindle 300 may insert or otherwise attach to a hinge 420 or wheel 410 of such structures on either side. For example, and referring to FIG. 13, spindles 300 are shown inserted into hinges 420 and wheels 410 of both left and right accordion-type structures.

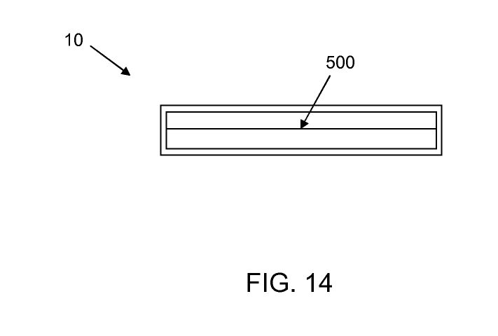

The cell culture apparatuses described herein may be used to culture any type of cell, such as adherent cell cultures and suspension cell cultures. While not shown, it will be understood that dialysis or transwell-type membranes may be included in a bag to separate the bag into two compartments or subchambers for biomolecule production, coculture, or the like. For example, and referring to FIG. 14, which depicts a longitudinal cross section of a bag 10 similar to the bag depicted in FIG. 1A. The bag 10 depicted in FIG. 14 includes a semi-permeable membrane 500 disposed in the bag 10. The semi-permeable membrane 500 divides the major chamber formed by the bag 10 into first and second compartments. One compartment may be used for cell culture, while the other may be used for (i) co-culture, (ii) obtaining biomolecules produced by the cultured cells, (iii) housing serum-free cell culture medium (e.g., while the cell culture compartment includes serum containing media), or the like. Any suitable semi-permeable membrane may be employed. Such membranes are readily available to those of skill in the art. The membrane 500 may be configured to selectively allow molecules having pre-determined characteristics to cross. For example, the membrane 500 may be configured to allow molecules of a particular molecular weight or size range to diffuse from the first compartment to the second compartment, while preventing such diffusion of molecules having a molecular weight or size range outside of the particular range. While not shown, it will be understood that separate ports may be employed to access each of the first and second compartments separately.

While the bulk of the disclosure herein describes culturing cells in substantially equal volumes of culture media within various subchambers, it will be understood that cells may be cultured in unequal volumes of culture medium or may be cultured in one or more subchambers and not cultured in one or more other subchambers. Whether the entire bag is used at one or whether only a part of the bag is used for culturing cells may depend on how tightly the bag segmentation is maintained at a particular fold.

Thus, embodiments of CELL CULTURE APPARATUS AND METHOD are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A method for culturing cells, the method comprising:
introducing culture media and cells into a flexible major culture chamber of a cell culture bag;
folding the bag to form first and second subchambers from the major chamber; and
incubating the cells in the first and second subchambers.

2. The method of claim 1, further comprising rotating the bag ninety degrees after folding the culture chamber back on itself to form the first and second subchambers.

3. The method of claim 3, wherein the bag is disposed within a housing having a first surface and an adjacent orthogonal second surface, and wherein rotating the bag ninety degrees comprises rotating the housing from a position resting on the first surface to a position resting on the second surface.

4. The method of claim 1, wherein the first chamber is fluidly sealed from the second chamber at a curved region where a portion of an internal surface of a first wall of the major chamber contacts a portion of an internal surface of an opposing second wall of the major chamber.

5. The method of claim 4, further comprising, prior to introducing the culture media and cells into the flexible major culture chamber, moving a portion of the first wall relative to the second wall in the curved region to allow fluid to flow between the region of the first chamber and the region of the second chamber.

6. The method of claim 5, wherein moving a portion of the first wall relative to the second wall comprises pulling the first wall.

7. The method of claim 6, further comprising ceasing the pulling of the wall to allow fluid sealing between the first and second subchambers.

8. The method of claim 7, wherein the application of the ceasing of the pulling occurs following substantially equal distribution of the introduced culture media and cells in the major chamber.

9. The method of claim 1, further comprising, prior to introducing the culture media and cells into the flexible major culture chamber, unfolding the bag to a sufficient degree to allow fluid to flow between the region of the first chamber and the region of the second chamber.

10. The method of claim 9, wherein folding the bag to form first and second subchambers occurs following substantially equal distribution of the introduced culture media and cells in the major chamber.

11. A cell culture apparatus comprising:
a housing;
a bag for culturing cells, the bag forming a major cell culture chamber and being disposed in the housing such that a second portion of the bag is folded relative to a first portion of the bag, thereby dividing the major chamber into first and second subchambers,
wherein the first portion of the bag forms the first subchamber and the second portion of the bag forms the second subchamber,
wherein the bag further includes a curved portion between the first and the second portions, the curved portion of the bag comprises a first wall and an opposing second wall, wherein a portion the first wall is moveable relative to a portion of the second wall from a position in contact with the second wall to fluidly seal the first subchamber from the second subchamber to a position away from the second wall to allow cell culture fluid to flow between the first and second subchambers.

12. The cell culture apparatus of claim 11, further comprising a pulling member configured to pull the portion of the first wall of the curved portion away from the portion of the second wall to allow fluid to flow between the first and second subchambers.

13. The cell culture apparatus of claim 12, wherein the pulling member comprises a body defining a lumen through which a vacuum may be applied.

14. A cell culture apparatus comprising:
a housing;
a bag for culturing cells, the bag forming a major cell culture chamber and being disposed in the housing such that a second portion of the bag is foldable and unfoldable relative to a first portion of the bag,
wherein when the bag is sufficiently folded (i) the major chamber is sealing divided into a first subchamber formed by the first portion of the bag and a second subchamber formed from the second portion of the bag, and (ii) the bag comprises a curved region disposed between the first and second portions of the bag;
a pivotable element aligned with the curved region of the bag, wherein pivoting of the pivotable element causes the bag to folded or unfolded.

15. The apparatus of claim 14, further comprising a spindle element operably couplable with the pivotable element, wherein the curved region of the bag is disposed about the spindle.

16. The apparatus of claim 14, wherein the housing comprises expandable and retractable side walls.

17. The apparatus of claim 16, wherein expansion of the side walls causes the pivotable element to pivot and cause the bag to unfold.

18. The apparatus of claim 16, wherein retraction of the side walls causes the pivotable element to pivot and cause the bag to fold.

19. The apparatus of claim 14, further comprising a semi-permeable membrane disposed in the bag, wherein the membrane divides the major cell culture chamber into first and second compartments and wherein the membrane is configured to selectively allow molecules having pre-determined characteristics to cross.
